(19) United States
(12) Patent Application Publication
(10) Pub. No.: US 2003/0208230 A1
(43) Pub. Date: Nov. 6, 2003

(54) Wrinkle free eye brows

(76) Inventor: Stanley D. C. Kim, Honolulu, HI (US)

Correspondence Address: Eugene OAK, Ph.D.J.D., Patent Attorney, 610 S. Van Ness Ave., Los Angeles, CA, 90005, US

(21) Appl. No.: 10/136,637

(22) Filed: May 2, 2002

(51) Int. Cl. A61F 5/08

(52) U.S. Cl. 606/204.35

(57) ABSTRACT

A novel solid sticker to unfold the vertical wrinkles between the eyebrows on human face. The solid sticker of this invention consists of solid support layer and adhesive sticker layer. The solid support of this invention is comprised of, including but not limited to, wood, plastic, rubber and metal to maintain the unfolded wrinkles. The exposed surface of the solid support has skin color, which is developed by, including, but not limited to, painting and covering with skin color tape. The adhesive sticker layer consists of a polymer film and adhesives coated on the one side thereof. The other side of polymer film is strongly adhered to the solid layer. The surface of adhesive coated layer, to be attached to the user's face, is covered with a peel off film. The peel is removed before use. The solid sticker of this invention unfolds the vertical eyebrow wrinkles on user's face when a user attaches it for at least 30 days, while replacing it every 24 hour. The sticker helps to improve a user's facial impression.

This invention relates to a novel solid sticker to unfold the vertical wrinkles between the eyebrows on a human face and improve a user's facial impression.

### FIELD OF THE INVENTION

A novel solid sticker of this invention consists of a solid support layer and an adhesive sticker layer. The solid sticker of this invention unfolds the vertical wrinkles on a user's face when a user attaches it for at least one month.

### DESCRIPTION OF THE PRIOR ARTS

Most of prior art for removing face wrinkles such as U.S. Pat. No. 5,582,585 and 5,116,675 to Nash-Morgan, and U.S. Pat. No. 4,658,811 Beaird teach to use flexible tape and adhesives to stretch the facial skin around the wrinkle to the opposite direction and fix them at both sides.

U.S. Pat. No. 3,949,741 to Hofmann teaches a method and appliance for reducing facial wrinkles by tightly applying a pressure-sensitive adhesive appliance to wrinkle prone areas of the human face in such a manner that the skin under the appliance is placed in a flattened and smoothed configuration. The appliance remains in place for at least four hours and then is removed in a manner to strip the dead skin cells from the skin. These steps are repeated a plurality of times over a long period of time. The appliance transmits moisture at least equal to that transmitted by facial skin of a human and is comprised of a film and a pressure-sensitive adhesive coating. The film has a tensile strength and an ultimate elongation consistent with flattening and smoothening the skin.

FIG. 1 is a Hoffmann's drawing of plastic film supported method for reducing facial wrinkles. The appliance 1 is composed of a plastic film 2 and an adhesive 3, coated thereon. The area of the skin to which the appliance is applied is indicated as 4. The appliance is in place the skin is in a generally planar configuration, as indicated by the centerline 5. When the appliance is pressed tightly onto that area, the adhesive locks into the wrinkle 6, as well as other portions of the skin. When pressure is released, after the appliance is tightly adhered to the skin, the adhesive draws the wrinkle upward towards the appliance, by virtue of the tight adhesion, and tends to flatten the wrinkle into a more planar and smooth configuration. If the thickness of the adhesive changes depending on the location, then the adhesive will fill the wrinkles. After that the polymer film should have a high resiliency to sustain the force to draw back of the adhesives from the wrinkled portion of the skin. If the film is not resilient enough, the film surface will collapse in to the wrinkle direction rather than draw back the wrinkled skin. However, the physical properties of the film of tensile strength, at least 3 pounds per inch, and an ultimate elongation of between 20 and 150% show that their film is flexible. In other words, the film also would be folded along the wrinkle unless another force sustains the wrinkle prone areas of a human face in such a manner that the skin under the appliance is placed in a flattened and smoothed configuration

### SUMMARY OF THE INVENTION

Therefore, it is the purpose of this invention to provide a novel solid sticker to unfold the vertical wrinkles between the eyebrows on a human face and maintain that surface structure for a desired time period. The solid sticker of this invention consists of solid support layer and an adhesive sticker layer. The solid support is comprised of, including but not limited to, solid material such as wood, plastic, and metal. The exposed surface of the solid support has skin color, which is developed by, including, but not limited to, painting and covering with skin colored tape. The adhesive sticker layer is comprised of a polymer film and adhesives coated one side thereof. The other side of polymer film is strongly adhered to the solid layer. The surface of adhesive coated layer, to be attached to the user's face, is covered with a peel off film. The peel is removed before use. The solid sticker of this invention unfolds the vertical wrinkles on user's face when the user attaches it during ordinary hours and while sleeping. The sticker helps to improve a user's facial impression.

### BRIEF DESCRIPTION OF THE DRAWINGS

FIG. 1 is a schematic drawing of prior plastic film supported method for reducing facial wrinkles.

FIG. 2 is a schematic drawing of solid supported sticker method for reducing facial wrinkles of this invention.

FIG. 3 is a cross sectional view of the solid sticker of this invention.

FIG. 4 is a schematic drawing of applying the solid sticker of this invention onto an eyebrow wrinkle.

### DETAILED DESCRIPTION OF THE PREFERRED EMBODIMENTS

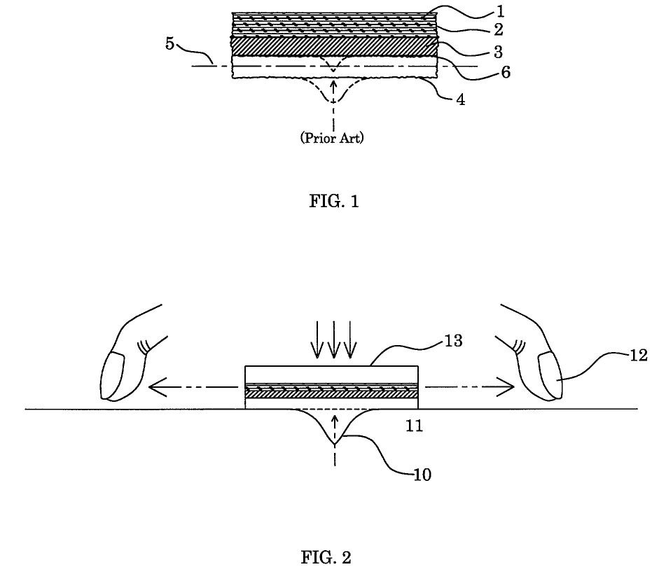

FIG. 1 is a schematic drawing of prior art's plastic film supported method for reducing facial wrinkles. The appliance 1 is composed of a plastic film 2 and an adhesive 3 coated thereon. The area 4 indicates the area of a skin to which the appliance is applied upon. The skin is in a generally planar configuration, as indicated by the centerline 5. When the appliance is pressed tightly onto that area, the adhesive locks into the wrinkle 6, as well as other portions of the skin. When pressure is released, after the appliance is tightly adhered to the skin, the adhesive draws the wrinkle upward towards the appliance, by virtue of the tight adhesion, and tends to flatten the wrinkle into a more planar and smooth configuration. The polymer film and adhesives must have high resiliency.

FIG. 2 is a schematic drawing of solid supported sticker method for reducing the facial wrinkles of this invention. The wrinkle (10) on the face skin (11) is stretched with fingers (12). The wrinkles will be stretched and the skin surface is moved upwards to the level of other skin surface. Subsequently attach the solid supported sticker of this invention (13) is attached on the stretched wrinkle surface (14) and pressed to maintain the flattened geometry. The sticker (13) is removed every 24 hour and repeats the procedure for one month. Then the wrinkle disappears. Experiment with the inventor's eyebrows wrinkle showed 100% removal of the wrinkle.

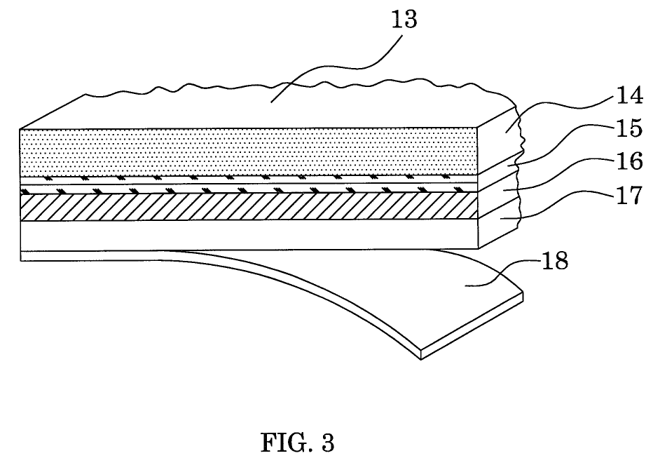

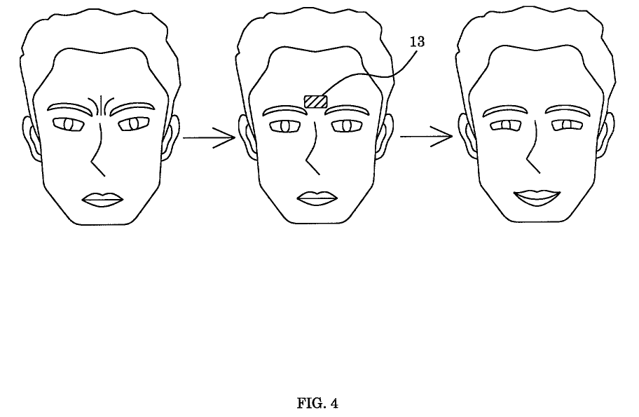

FIG. 3 is a cross sectional view of the solid sticker of this invention. The support (14) is a solid material such as wood and metal or highly resilient material such as plastic and rubber or blend of the two. The thickness of the support layer is at least 0.5 cm to keep the solidity and resiliency. The support layer (14) is adhered to a polymer film (16) with strong adhesive (15) such as epoxy resin adhesives. The other side of the polymer film (16) is coated with a relatively weak and non-allergenic adhesive (17) for a human skin. The coated adhesive layer is protected by the peel (18) easily removable prior to use.

FIG. 4 is a schematic drawing of the effect of the applying of the solid sticker of this invention onto an eyebrow wrinkle. The facial impression changes to a more bright and happier one.

The best mode of this invention is to accomplish when a wood and elastic plastic plate as the support are used. Moisturizing components impregnated in the adhesive layer (17) facilitates unfolding of the wrinkles.

What is claimed is:

1. A novel solid sticker consists of a solid support layer comprised of wood, plastic, rubber and metal, thickness of the solid support layer, being in the range of 0.1 cm to 0.5 cm, and an adhesive sticker layer consisting of a polymer film, one side of which being strongly adhered to the solid support layer and the other side of which being coated with adhesives for a user's skin, covered with peel off film, to unfold the vertical wrinkles between the eyebrows on human face.

2. The exposed surface of the solid support layer, in claim 1, has skin color, which is developed by a painting and a covering with skin colored tape.

3. The solid sticker, in claim 1, unfolds the vertical eyebrow wrinkles on user's face when a user attaches it at least 30 days while replacing it every 24 hours.
